(19) United States
(12) Patent Application Publication
(10) Pub. No.: US 2024/0130787 A1
(43) Pub. Date: Apr. 25, 2024

(54) COAXIAL TIME-OF-FLIGHT OPTICAL FIBER DISTANCE MEASUREMENT USING DUAL COMB RANGING

(71) Applicant: GYRUS ACMI, INC. D/B/A OLYMPUS SURGICAL TECHNOLOGIES AMERICA, Westborough, MA (US)

(72) Inventors: Charles A. Baker, Rogers, MN (US); Kester Julian Batchelor, Mound, MN (US)

(21) Appl. No.: 18/477,062

(22) Filed: Sep. 27, 2023

Related U.S. Application Data
(60) Provisional application No. 63/380,176, filed on Oct. 19, 2022.

(51) Int. Cl. A61B 18/22

(52) U.S. Cl. CPC A61B 18/22; A61B 2018/00982

(57) ABSTRACT

An optical fiber having a distal end extending from a distal end of an endoscope can direct light to and from a target. An interferometer can receive first light pulses from a first frequency comb having a first repetition rate, form reference arm light pulses and measurement arm light pulses from the first light pulses, direct the measurement arm light pulses to and from the target via the optical fiber to form return light pulses, and interfere the return light pulses with the reference arm light pulses to form interferometer output pulses. A beamsplitter can interfere the interferometer output pulses with second light pulses from a second frequency comb having a second repetition rate to form system output pulses. Processor circuitry can determine, from a time duration between consecutive system output pulses, a spacing between the optical fiber and the target, and can take an action in response.

### CROSS-REFERENCE TO RELATED APPLICATION

This application claims the benefit of U.S. Provisional Application No. 63/380,176, filed Oct. 19, 2022, which is hereby incorporated by reference in its entirety.

### FIELD OF THE DISCLOSURE

This document relates generally to endoscopic systems, and more specifically relates to systems and methods for determining and controlling a distance between an endoscope tip and a target.

### BACKGROUND OF THE DISCLOSURE

An operator, such as a physician, practitioner, or user, can use an endoscope to provide visual access to an internal location of a patient. The operator can insert an endoscope into a patient's body. The endoscope can deliver light to a target being examined, such as a target anatomy or object. The endoscope can collect light that is reflected from the object. The reflected light can carry information about the target being examined.

An endoscope can include a working channel. In some examples, the operator can perform suction through the working channel. In some examples, the operator can pass instruments, such as brushes, biopsy needles or forceps, through the working channel. In some examples, the operator can perform minimally invasive surgery through the working channel, such as to remove unwanted tissue or foreign objects from the body of the patient.

An endoscope can use a laser or plasma system to perform laser therapy, such as ablation, coagulation, vaporization, fragmentation, lithotripsy, and others. In laser therapy, the operator can use the endoscope to deliver surgical laser energy to various target treatment areas, such as soft or hard tissue. In lithotripsy, the operator can use the endoscope to deliver surgical laser energy to break down calculi structures in the patient's kidney, gallbladder, ureter, or other stone-forming regions, or to ablate large calculi into smaller fragments.

### SUMMARY

In an example, an endoscopic system can comprise: an optical fiber having a distal end extending from a distal end of an endoscope and configured to direct light to and from a target; an interferometer configured to: receive first light pulses from a first frequency comb having a first repetition rate; form reference arm light pulses and measurement arm light pulses from the first light pulses; direct the measurement arm light pulses to and from the target via the optical fiber to form return light pulses; and interfere the return light pulses with the reference arm light pulses to form interferometer output pulses; a beamsplitter configured to interfere the interferometer output pulses with second light pulses from a second frequency comb having a second repetition rate different from the first repetition rate to form system output pulses; an optical detector configured to sense the system output pulses; and processor circuitry configured to: determine, from a time duration between consecutive system output pulses, a spacing between the distal end of the optical fiber and the target; and generate a spacing data signal representing the determined spacing.

In an example in which an endoscopic system includes an optical fiber having a distal end extending from a distal end of an endoscope, a method for operating the endoscopic system can comprise: receiving, with an interferometer, first light pulses from a first frequency comb having a first repetition rate; forming, with the interferometer, reference arm light pulses and measurement arm light pulses from the first light pulses; directing the measurement arm light pulses to and from a target via the optical fiber to form return light pulses; interfering the return light pulses with the reference arm light pulses to form interferometer output pulses; interfering, with a beamsplitter, the interferometer output pulses with second light pulses from a second frequency comb having a second repetition rate different from the first repetition rate to form system output pulses; sensing, with an optical detector, the system output pulses; determining, with processor circuitry, from a time duration between consecutive system output pulses, a spacing between the distal end of the optical fiber and the target; and generating, with the processor circuitry, a spacing data signal representing the determined spacing.

In an example, an endoscopic system can comprise: an endoscope; a therapeutic laser light source spaced apart from the endoscope and configured to generate therapeutic light pulses at first times; a first frequency comb spaced apart from the endoscope and configured to generate first light pulses that repeat at a first repetition rate; a Michelson interferometer configured to split the first light pulses between a reference arm and a measurement arm to form respective reference arm light pulses that repeat at the first repetition rate and measurement arm light pulses that repeat at the first repetition rate; an optical fiber including a distal end extending from the endoscope, the optical fiber configured to: receive the therapeutic light pulses at the first times; receive the measurement arm light pulses at second times different from the first times; direct the therapeutic light pulses and the measurement arm light pulses along the optical fiber to emerge from the distal end of the optical fiber toward a target; collect, as collected light pulses, at least some of the measurement arm light pulses that are reflected from the target; and direct, as return light pulses, at least some of the collected light pulses along the optical fiber away from the distal end of the optical fiber, the Michelson interferometer further configured to interfere the return light pulses with the reference arm light pulses to form interferometer output pulses; a second frequency comb spaced apart from the endoscope and configured to generate second light pulses at a second repetition rate different from the first repetition rate; a beamsplitter configured to interfere the interferometer output pulses with the second light pulses to form system output pulses; an optical detector configured to sense the system output pulses; processor circuitry configured to: determine, from a time duration between consecutive system output pulses, a spacing between the distal end of the optical fiber and the target; and generate a spacing data signal representing the determined spacing; an illumination light source disposed at a distal end of the endoscope and configured to illuminate the target with visible illumination light; a camera disposed at the distal end of the endoscope and configured to generate a video image of the illuminated target; and a display coupled to the processor circuitry and configured to display the video image of the illuminated target and a visual representation of the determined spacing represented by the spacing data signal.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 shows a side-view schematic drawing of an example of an endoscopic system.

FIG. 2 shows a flow chart of an example of a method for operating an endoscopic system.

FIG. 3 shows a schematic diagram of an example of a computer-based clinical decision support system (CDSS) that is configured to determine a spacing between a distal end of an optical fiber and a target, and, in response, generate a spacing data signal and/or take a suitable action.

### DETAILED DESCRIPTION

In a laser therapy treatment, a practitioner can position a distal end of an endoscope close to a target, such as a kidney stone. The endoscope can include an optical fiber that can deliver therapeutic laser light to the target, such as via a distal end of the optical fiber, to ablate tissue that is at or near the distal end of the optical fiber. During a procedure, the tissue can absorb the laser light, can heat locally to a relatively high temperature, and can break apart due to local thermal strains within the tissue.

During the treatment, it can be beneficial to dynamically monitor or dynamically control a separation between the distal end of the optical fiber and the target. For example, if the distal end of the optical fiber is positioned too close to the target, a condition known as flashing may occur, which can degrade the distal end of the optical fiber. Likewise, if the distal end of the optical fiber is positioned too far from the target, then a significant fraction of the therapeutic laser light can be absorbed before reaching the target, which can decrease an efficiency of the laser therapy treatment or cause the treatment to take longer.

The endoscopic system described in detail below can use dual comb ranging techniques on light that returns through the optical fiber to dynamically monitor a real-time separation between the distal end of the optical fiber and the target. Because the measurement technique uses light that returns through the optical fiber, the measurement technique can be referred to as being coaxial.

Specifically, during a laser therapy treatment, the endoscopic system can use dual comb ranging techniques on light that returns through the optical fiber to dynamically determine the real-time separation, and, in response to the real-time separation value, can provide user feedback and/or take an action. For example, the endoscopic system can provide user feedback representing the real-time separation to the practitioner, such as displaying a numerical value on a display, displaying a graphical representation of the real-time separation on a display, displaying visual indicators that show when the real-time separation is in one of several specified ranges (such as too small, acceptable, too large, and so forth), playing an audio alert, and others. As another example, the endoscopic system can take an action in response to the real-time separation, such as proximally retracting the optical fiber if the real-time separation is too low, automatically positioning the distal end of the optical fiber to have a specified value of real-time separation, or others.

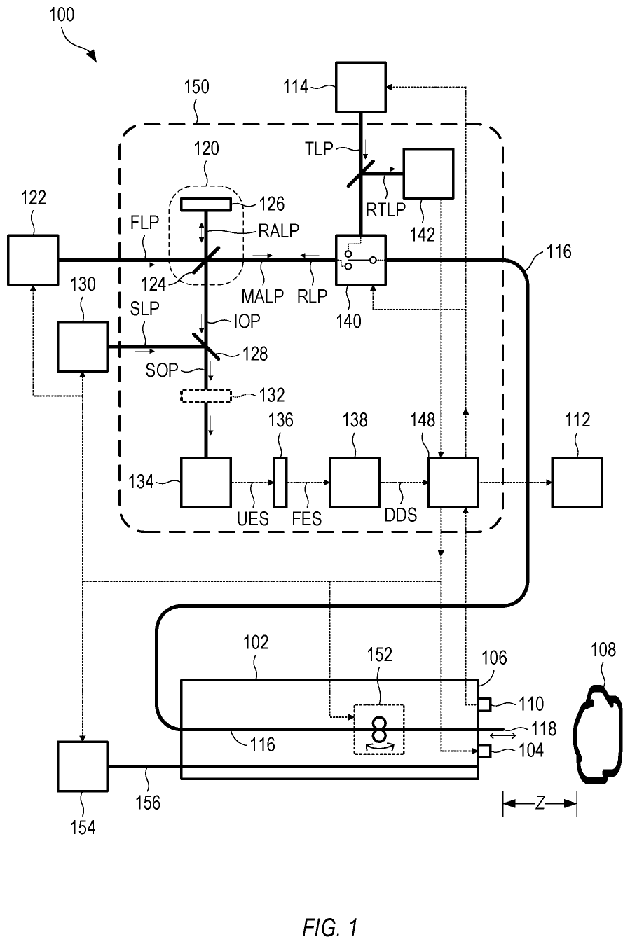

FIG. 1 shows a side-view schematic drawing of an example of an endoscopic system 100. The endoscopic system 100 can include an endoscope 102. The endoscope 102 can be grippable by the operator, who can position the endoscope 102 as needed to view and ablate one or more targets, such as kidney stones, in one or more internal locations of the patient. In some examples, the endoscope 102 can be rigid. In one or more examples, the endoscope 102 can be elongated along an elongation axis. The endoscope 102 can include one or more channels, passages, or apertures that extend through the endoscope 102 along the elongated axis. For example, the endoscope 102 can include a working channel. In some examples, the operator can perform suction through the working channel. In some examples, the operator can pass instruments, such as brushes, biopsy needles or forceps, through the working channel. In some examples, the operator can perform minimally invasive surgery through the working channel, such as to remove unwanted tissue or foreign objects from the body of the patient. As another example, the endoscope 102 can include an irrigation channel, which can supply irrigant to the target 108, such as to flush away pieces of the target. Other channels can also be used.

The endoscopic system 100 can include an illumination light source 104 disposed on a distal end 106 of the endoscope 102. For example, the illumination light source 104 can include one or more light emitting diodes disposed on the distal end 106 of the endoscope 102. In some examples, the light emitting diodes can be white light emitting diodes. For example, a white light emitting diode can include a blue or a violet light emitting diode, coupled with a phosphor that can absorb some or all of the blue or violet light, and in response, can emit light with one or more longer wavelengths, such as in the yellow portion of the electromagnetic spectrum. Other illumination light sources can also be used. The illumination light source 104 can illuminate a target 108 with visible illumination light having a visible illumination light spectral range. In some examples, the visible illumination light spectral range can include wavelengths in the visible portion of the electromagnetic spectrum.

The endoscopic system 100 can include a camera 110, such as a video camera, disposed on the distal end 106 of the endoscope 102. In some examples, the camera 110 can include a lens, a sensor element located at a focal plane of the lens, and electronics that can convert an electrical signal produced by the sensor element into a digital signal. The camera elements can be located in a relatively small, sealed package at the distal end 106 of the endoscope 102. The camera 110 can capture, or generate, a real-time video image of the illuminated target 108.

The endoscopic system 100 can include a display 112, such as a video display, that can display the video image of the illuminated target 108. For example, the display 112 can be mounted on or in a rack of equipment, away from the endoscope 102, and separate from a housing 150 that can surround most or all of the components that are not light sources. The display 112 can provide, or display, a real-time video image of the target 108, illuminated with white light from the illumination light source 104, to the practitioner. In some examples, the display 112 can be coupled to processor circuitry 148 (described below). In some examples, the display 112 can be configured to display the video image of the illuminated target and a visual representation of a spacing between a distal end 118 of an optical fiber 116 and the target 108. For example, the visual representation can include one or more of: an alphanumeric display of the spacing, a graphical display of the spacing, such as on a dial, one or more colors that represent the spacing with respect to one or more specified ranges of spacings, such as a display of the color green to indicate that the spacing is in an acceptable range, a display of the color red to indicate that the spacing is in an unacceptable range, and so forth. Other display schemes can also be used.

The endoscopic system 100 can include a therapeutic laser light source 114 that can generate laser light, such as in pulsed laser light. The therapeutic laser light source 114 can be located away from the endoscope 102, such that the endoscope 102 can be positionable by the operator, while the therapeutic laser light source 114 can be disposed in a laser housing that can remain in a fixed position, spaced apart from the endoscope 102, during a procedure. In some examples, the therapeutic laser light source 114 can include a thulium fiber laser, which can produce light having one or more wavelengths between about 1920 nm and about 1960 nm. In some examples, the therapeutic laser light source 114 can include a thulium:YAG (yttrium aluminum garnet) laser, which can produce light at a wavelength of 2010 nm. In some examples, the therapeutic laser light source 114 can include a holmium:YAG laser, which can produce light at a wavelength of 2120 nm. In some examples, the therapeutic laser light source 114 can include an erbium:YAG laser, which can produce light at a wavelength of 2940 nm. In some examples, the laser light produced by the therapeutic laser light source 114 can include a first wavelength, such as a wavelength between about 1908 nm and about 2940 nm, or between about 1920 nm and 1960 nm, between about 1900 nm and about 1940 nm, greater than about 1900 nm, greater than about 1800 nm, or others. For these (and other) laser light sources, the laser light can have a wavelength or wavelengths in a portion of the electromagnetic spectrum at which water (a major component of tissue) has a relatively high absorption. During a procedure, the tissue can absorb the laser light, can heat locally to a relatively high temperature, and can break apart due to local thermal strains within the tissue.

The endoscopic system 100 can include an optical fiber 116 that can extend from the endoscope 102. In some examples, the optical fiber 116 can be a multi-mode optical fiber. In some examples, the optical fiber 116 can have a distal end 118 that extends from a distal end 106 of the endoscope 102. In some examples, the optical fiber 116 can direct light to and from the target 108.

In some examples, the optical fiber 116 can collect, as collected therapeutic light pulses (not shown), at least some of the therapeutic light pulses (TLP) that are reflected from the target 108. In some examples, the optical fiber 116 can direct, as return therapeutic light pulses (RTLP), at least some of the collected therapeutic light pulses along the optical fiber 116 away from the distal end 118 of the optical fiber 116.

In some examples, the endoscopic system 100 can include a spectrometer 142 that can analyze the return therapeutic light pulses. For example, the endoscopic system 100 can use the spectrometer 142 to perform analysis of the target 108, based on a spectrum of the return therapeutic light pulses (RTLP). For example, the spectrometer 142 and the processor circuitry 148 (described below) can use the spectral profile of the target 108 to determine a material composition of the target 108, such as by matching the measured spectral profile of the target 108 to one or more of a specified (finite) plurality of predetermined spectral profiles that correspond to known materials. These are but examples; other suitable analyses of the target 108 can also be performed. The spectrometer 142 can generate a spectrometer output signal that includes data that represents light intensity (or amplitude, or other suitable photometric quantity) as a function of wavelength. The processor circuitry 148 (described below) can receive and interpret the spectrometer output signal.

In some examples, the optical fiber 116 can be time-multiplexed between delivering light that is used for therapy (e.g., the high-powered light that is absorbed by the target 108 and physically ablates the target 108) and delivering light that is used to determine a real-time separation between the distal end 118 of the optical fiber 116 and the target 108. The separation determination is described in detail presently.

The technique for determining the real-time separation between the distal end 118 of the optical fiber 116 and the target 108 can be referred to as dual comb ranging. Dual comb ranging can use light from two optical frequency combs that have slightly different repetition rates. Each optical frequency comb can be a broadband coherent light source that includes a series of discrete longitudinal optical modes. Each optical mode can be described in terms of a repetition rate (fᵣ) and an offset frequency (fₒ), as f(n)=nfᵣ+fₒ.

The endoscopic system 100 can include an interferometer 120, such as a Michelson interferometer. The interferometer 120 can receive first light pulses (FLP) from a first frequency comb 122 having a first repetition rate. The interferometer 120 can form reference arm light pulses (RALP) and measurement arm light pulses (MALP) from the first light pulses (FLP), such as by splitting the first light pulses (FLP) with a beamsplitter 124. The interferometer 120 can include a reference arm reflector 126, such as a mirror, that can reflect the reference arm light pulses (RALP) back toward the beamsplitter 124.

The interferometer 120 can direct the measurement arm light pulses (MALP) to and from the target 108 via the optical fiber 116 to form return light pulses (RLP). For example, the optical fiber 116 can be configured such that the measurement arm light pulses (MALP) enter the optical fiber 116, propagate to the distal end 118 of the optical fiber 116, emerge from the distal end 118 of the optical fiber 116, reflect from the target 108, enter the distal end 118 of the optical fiber 116, propagate away from the distal end 118 of the optical fiber 116, and exit the optical fiber 116 to form the return light pulses (RLP). The measurement arm light pulses (MALP) can be temporally offset from the corresponding reference arm light pulses (RALP) by a time interval that varies as a function of the spacing between the distal end 118 of the optical fiber 116 and the target 108.

The interferometer 120 can interfere the return light pulses (RLP) with the reference arm light pulses (RALP) to form interferometer output pulses (TOP). Before interference, the reference arm light pulses (RALP) experience a round-trip time-of-flight delay equal to twice the distance between the reference arm reflector 126 and the beamsplitter 124. Before interference, the return light pulses (RLP) experience a round-trip time-of-flight delay that includes a round-trip time-of-flight delay between the distal end 118 of the optical fiber 116 and the target 108, plus a round-trip time-of-flight delay between the beamsplitter 124 and the distal end 118 of the optical fiber 116.

The endoscopic system 100 can include a beamsplitter 128 that can interfere the interferometer output pulses (TOP) with second light pulses (SLP) from a second frequency comb 130 having a second repetition rate different from the first repetition rate to form system output pulses (SOP). In some examples, the first frequency comb and the second frequency comb can be spaced apart from the endoscope. In some examples, the first light pulses and the second light pulses can be spectrally separated from the therapeutic light pulses (e.g., such that a wavelength-sensitive beamsplitter can separate the comb light from the therapeutic light and/or combine the comb light and the therapeutic light.

The endoscopic system 100 can optionally include an optical bandpass filter 132 that can reduce an optical spectrum of the system output pulses (SOP), such as by filtering out aliasing and/or undesired higher harmonics. In some examples, the optical bandpass filter 132 can be tunable.

The endoscopic system 100 can include an optical detector 134 that can sense the system output pulses (SOP). The optical detector 134 can include one or more light-sensitive sensor elements, which can convert an optical signal, such as the system output pulses (SOP), into an internal electrical signal. In some examples, the optical detector 134 can generate an unfiltered electrical signal (UES) in response to the sensed system output pulses.

The endoscopic system 100 can optionally include a low-pass filter 136 that can reduce high frequency content of the unfiltered electrical signal (UES) to form a filtered electrical signal (FES), such as by filtering out and/or attenuating frequencies above a specified cutoff frequency.

The endoscopic system 100 can include an analog-to-digital converter 138 and accompanying sensor circuitry (not shown) that can receive the filtered electrical signal (FES) and, in response, generate a digital detector signal (DDS).

The endoscopic system 100 can include processor circuitry 148. In some examples, the processor circuitry 148 may be referred to as a controller. In some examples, the processor circuitry 148 may be implemented purely in software. In some examples, the processor circuitry 148 may be implemented purely in hardware. In some examples, the processor circuitry 148 may be implemented as a combination of software and hardware. In some examples, the processor circuitry 148 may be implemented on a single processor. In some examples, the processor circuitry 148 may be implemented on multiple processors. In some examples, the multiple processors may be housed in a common housing. In some examples, at least two of the multiple processors may be spaced apart in different housings.

The processor circuitry 148 can analyze the digital detector signal (DDS) to determine a time duration between consecutive system output pulses (SOP). The processor circuitry 148 can determine, from the time duration between consecutive system output pulses (SOP), a spacing between the distal end 118 of the optical fiber 116 and the target 108. For example, the processor circuitry 148 can determine the spacing to equal one-half of a product of the time duration between consecutive system output pulses (SOP), an optical pulse velocity in the (liquid) medium between the distal end 118 of the optical fiber 116 and the target 108, and a difference between the first repetition rate and the second repetition rate, divided by the first repetition rate. This is but one example of a technique for determining the spacing from the measured time duration; other determination techniques can also be used. The processor circuitry 148 can generate a spacing data signal representing the determined spacing.

In some examples, the optical fiber 116 can be time-multiplexed to deliver the measurement arm light pulses (MALP) to and from the target 108 at first times and deliver the therapeutic light pulses (TLP) at second times different from the first times. The therapeutic light pulses (TLP) can ablate the target. In some examples, the therapeutic light pulses (TLP) can be spectrally separated from the first light pulses (FLP) and/or spectrally separated from the second light pulses (SLP). To facilitate the time-multiplexing, the endoscopic system 100 can include a time-multiplexer 140. Although the time-multiplexer 140 is shown in FIG. 1 as being a switch that can be electrically controlled or actuated by the processor circuitry 148, many time-multiplexing schemes are possible. For example, the time-multiplexer 140 can be executed in software, such as the processor circuitry 148 causing the therapeutic laser light source 114, the first frequency comb 122, and the second frequency comb 130 to switch on and off at suitable times. As another alternative, the time-multiplexer 140 can employ one or more wave-sensitive optical elements to combine and/or separate the therapeutic light from the separation-measuring light. Other time-multiplexing schemes can also be used.

During a laser therapy treatment, the endoscopic system 100 can use dual comb ranging techniques on light that returns through the optical fiber 116 to dynamically determine the real-time separation, and, in response to the real-time separation value, can take an action.

An example of an action (taken in response to comparing the separation to a threshold separation value) is to dynamically adjust the separation (e.g., by dynamically varying or adjusting the distance (Z) in FIG. 1). For example, the endoscopic system 100 can further include an actuator 152 that can advance the optical fiber 116 distally and retract the optical fiber 116 proximally with respect to the endoscope 102. The processor circuitry 148 can compare the determined spacing to a specified threshold and cause the actuator 152 to automatically reduce a difference between the determined spacing and the specified threshold. Doing so can increase an efficiency of the procedure and may help prevent damage to the optical fiber 116, such as might be caused by a flashing event that may occur if the spacing were too small.

In some examples, such as the configuration of FIG. 1, the actuator 152 can include a wheel. The wheel can have a center that is fixed in position with respect to the endoscope 102. The wheel can have a circumferential surface that contacts the optical fiber 116. The wheel can be rotatable from a rotary actuator, such as a rotary actuator disposed at or near the center of the wheel. In some examples, the actuator 152 can automatically retract the optical fiber 116 proximally with respect to the endoscope 102 by a specified distance in response to receiving the data that indicates that the distal end 118 of the optical fiber 116 is too close to the target 108. The actuator 152 described above and shown in FIG. 1 is but one example of an actuator that can advance the optical fiber 116 distally and retract the optical fiber 116 proximally with respect to the endoscope 102. Other suitable actuators can also be used.

Another example of an action (taken in response to determining that the spacing represented by the spacing data signal is less than a specified threshold spacing) is to cause the therapeutic laser light source 114 to reduce its output power, optionally to zero.

Another example of an action (taken in response to determining that the spacing represented by the spacing data signal is less than a specified threshold spacing) is to supply more irrigant to the target 108. For example, the endoscopic system 100 can include an irrigation regulator 154 coupled to the endoscope. The irrigation regulator 154 can supply an irrigant, such as a saline solution, to the target 108, via an irrigation line 156, at a controllable irrigation rate. The processor circuitry 148 can cause the irrigation regulator 154 to increase the irrigation rate in response to receiving data that indicates that the distal end 118 of the optical fiber 116 is too close to the target 108.

Another example of an action (taken in response to determining that the spacing represented by the spacing data signal is less than a specified threshold spacing) is to suppress or interrupt a displayed video image of the target 108 that is displayed on the display 112. The processor circuitry 148 can suppress the video image in response to receiving data that indicates that the distal end 118 of the optical fiber 116 is too close to the target 108. The examples of actions taken in response to the determined spacing are but mere examples; the processor circuitry 148 can alternatively cause other suitable actions to occur.

Note that in FIG. 1, any or all of the optical paths between optical elements can either involve free space propagation, such as using a collimating lens or focusing lens to form a collimated beam in free space, fiber propagation, such as propagating using a multimode fiber or single-mode fiber, or a combination of both free space propagation and fiber propagation. The collimating lenses or focusing lenses are omitted from FIG. 1 for clarity.

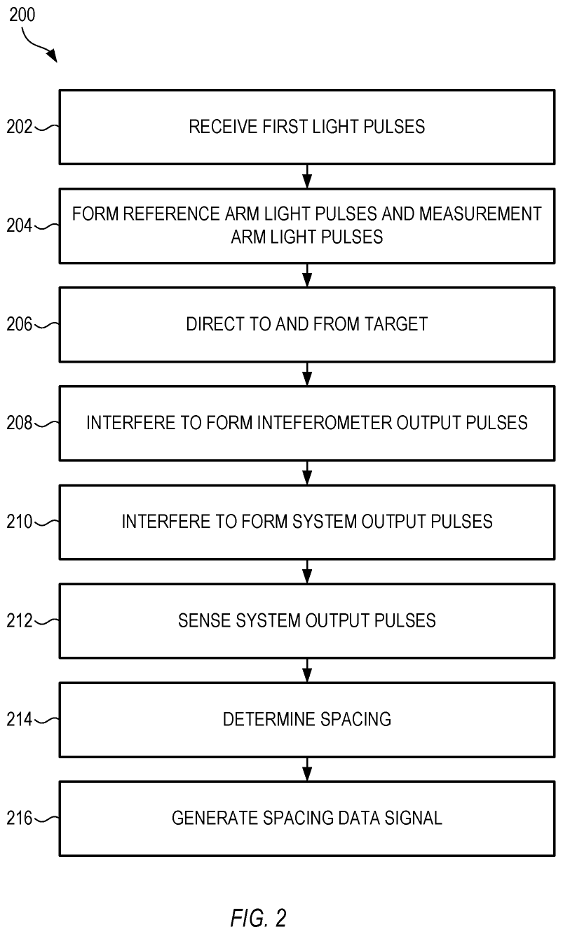

FIG. 2 shows a flow chart of an example of a method 200 for operating an endoscopic system, such as the endoscopic system 100 of FIG. 1, or any other suitable endoscopic system. The method 200 is but one example of a method for operating a endoscopic system; other methods can also be used. The endoscopic system can include an optical fiber having a distal end extending from a distal end of an endoscope.

At operation 202, an interferometer can receive first light pulses from a first frequency comb having a first repetition rate.

At operation 204, the interferometer can form reference arm light pulses and measurement arm light pulses from the first light pulses.

At operation 206, the interferometer can direct the measurement arm light pulses to and from a target via the optical fiber to form return light pulses.

At operation 208, the interferometer can interfere the return light pulses with the reference arm light pulses to form interferometer output pulses.

At operation 210, a beamsplitter can interfere the interferometer output pulses with second light pulses from a second frequency comb having a second repetition rate different from the first repetition rate to form system output pulses.

At operation 212, an optical detector can sense the system output pulses.

At operation 214, processor circuitry can determine, from a time duration between consecutive system output pulses, a spacing between the distal end of the optical fiber and the target.

At operation 216, the processor circuitry can generate a spacing data signal representing the determined spacing.

In some examples, the endoscopic system can further include a therapeutic laser light source spaced apart from the endoscope and configured to generate the therapeutic light pulses at the second times.

In some examples, the optical fiber can be time-multiplexed to deliver the measurement arm light pulses to and from the target at first times and deliver the therapeutic light pulses at second times different from the first times.

In some examples, the therapeutic light pulses are configured to ablate the target.

In some examples, the therapeutic light pulses are spectrally separated from the first light pulses.

In some examples, the method 200 can optionally further include using the processor circuitry to vary at least one operational parameter of the therapeutic laser light source in response to the determined spacing represented by the spacing data signal.

In some examples, the method 200 can optionally further include using the processor circuitry to automatically switch off the therapeutic laser light source when the determined spacing represented by the spacing data signal is less than a specified threshold spacing.

In some examples, the endoscopic system can further include an actuator configured to advance the optical fiber distally and retract the optical fiber proximally with respect to the endoscope. In some examples, the method 200 can optionally further include using the processor circuitry to compare the determined spacing to a specified threshold. In some examples, the method 200 can optionally further include using the processor circuitry to cause the actuator to automatically reduce a difference between the determined spacing and the specified threshold.

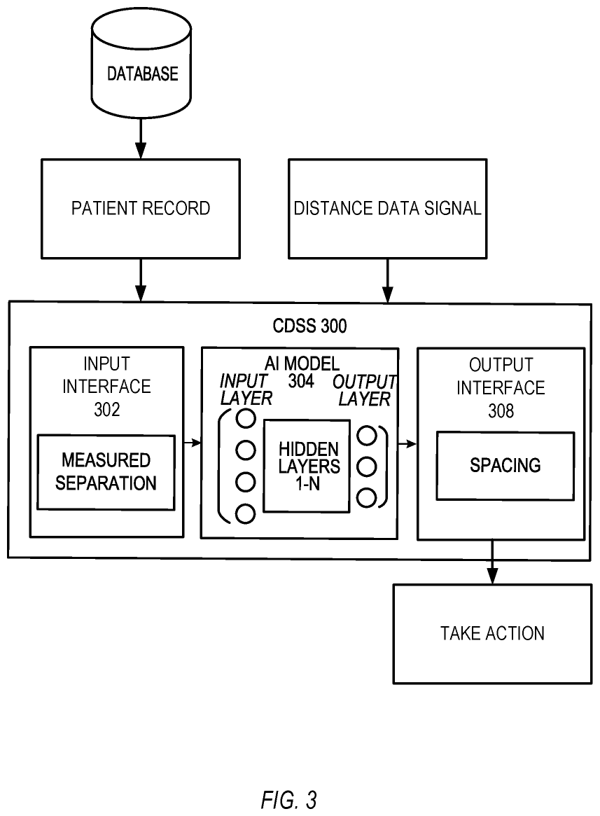

FIG. 3 shows a schematic diagram of an example of a computer-based clinical decision support system (CDSS) 300 that is configured to determine a spacing between a distal end of an optical fiber and a target, and, in response, generate a spacing data signal and/or take a suitable action, such as automatically distally advance or automatically proximally retract the optical fiber. In various embodiments, the CDSS 300 includes an input interface 302 through which the spacing data signal can be provided as input features to an artificial intelligence (AI) model 304, a processor such as a controller or processor circuitry 148 which performs an inference operation in which the determined spacing can be communicated to a user, e.g., a clinician.

In some embodiments, the input interface 302 may be a direct data link between the CDSS 300 and one or more medical devices, such as endoscopic system 100 or endoscope, which generate at least some of the input features. For example, the input interface 302 may transmit the determination directly to the CDSS during a therapeutic and/or diagnostic medical procedure. Additionally, or alternatively, the input interface 302 may be a classical user interface that facilitates interaction between a user and the CDSS 300. For example, the input interface 302 may facilitate a user interface through which the user may manually enter the determination. Additionally, or alternatively, the input interface 302 may provide the CDSS 300 with access to an electronic patient record from which one or more input features may be extracted. In any of these cases, the input interface 302 is configured to collect the determination in association with a specific patient on or before a time at which the CDSS 300 is used to assess the medical condition addressed by the endoscopic system 100 or endoscope, such as a kidney stone.

Based on one or more of the above input features, the controller or processor circuitry 148 performs an inference operation using the AI model to generate the determination. For example, input interface 302 may deliver the spacing data signal into an input layer of the AI model which propagates this input feature through the AI model to an output layer. The AI model can provide a computer system the ability to perform tasks, without explicitly being programmed, by making inferences based on patterns found in the analysis of data. AI model explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model may be trained continuously or periodically prior to performance of the inference operation by the controller or processor circuitry 148. Then, during the inference operation, the patient specific input features provided to the AI model may be propagated from an input layer, through one or more hidden layers, and ultimately to an output layer that corresponds to the value of spacing or distance (Z).

In some examples, the AI model can include a database, which can include data corresponding to a patient. The database can provide a patient record to the CDSS 300.

During and/or subsequent to the inference operation, the determination may be communicated to the user via an output user interface 308 and/or automatically cause an actuator or an alarm connected to the processor to perform a desired action. For example, the controller or processor circuitry 148 can cause an actuator to move the optical fiber with respect to the endoscope. Alternatively, the controller or processor circuitry 148 can cause an alarm to alert the practitioner. In some examples, the CDSS 300 can optionally be used to determine the action taken in response to a spacing data signal.

Some features as described herein may provide methods and apparatus that can identify the composition of various targets, for instance, in medical applications (e.g., soft or hard tissue) in vivo through an endoscope. This may allow the operator to continuously monitor the composition of the target viewed through the endoscope throughout the procedure. This also can be used in combination with a laser system where the method may send feedback to the laser system to adjust the settings based on the composition of the target. This feature may allow for the instant adjustment of laser settings within a set range of the original laser setting selected by the operator.

Some features as described herein may be used to provide a system and method that measures differences, such as the chemical composition of a target, in vivo and suggests laser settings or automatically adjusts laser settings to better achieve a desired effect. Examples of targets and applications include laser lithotripsy of renal calculi and laser incision or vaporization of soft tissue. In one example, three major components are provided: the laser, the spectroscopy system, and the feedback analyzer. In an example, a controller of the laser system may automatically program laser therapy with appropriate laser parameter settings based on target composition. In an example, the laser may be controlled based on a machine learning algorithm trained with spectroscope data. Additionally or alternatively, an operator may receive an indication of target type continuously during the procedure, and be prompted to adjust the laser setting. By adjusting laser settings and adapting the laser therapy to composition portions of a single calculus target, stone ablation or dusting procedures can be performed faster and in a more energy-efficient manner.

Some features as described herein may provide systems and methods for providing data inputs to the feedback analyzer to include internet connectivity, and connectivity to other surgical devices with a measuring function. Additionally, the laser system may provide input data to another system such as an image processor whereby the procedure monitor may display information to the operator relevant to the medical procedure. One example of this is to identify different soft tissues more clearly in the field of view during a procedure, vasculature, capsular tissue, and different chemical compositions in the same target, such as a stone for example.

Some features as described herein may provide systems and methods for identifying different target types, such as different tissue types, or different calculus types. In some cases, a single calculus structure (e.g., a kidney, bladder, pancreobiliary, or gallbladder stone) may have two or more different compositions throughout its volume, such as brushite, calcium phosphate (CaP), dihydrate calcium oxalate (COD), monohydrate calcium oxalate (COM), magnesium ammonium phosphate (MAP), or a cholesterol-based or a uric acid-based calculus structure. For example, a target calculus structure may include a first portion of COD and a second portion of COM. According to one aspect, the present document describes a system and a method for continuously identifying different compositions contained in a single target (e.g., a single stone) based on continuous collection and analysis of spectroscopic data in vivo. The treatment (e.g., laser therapy) may be adapted in accordance with the identified target composition. For example, in response to an identification of a first composition (e.g., COD) in a target stone, the laser system may be programmed with a first laser parameter setting (e.g., power, exposure time, or firing angle, etc.) and deliver laser beams accordingly to ablate or dust the first portion. Spectroscopic data may be continuously collected and analyzed during the laser therapy. In response to an identification of a second composition (e.g., COM) different than the first composition in the same target stone being treated, the laser therapy may be adjusted such as by programming the laser system with a second laser parameter setting different from the laser parameter setting (e.g., different power, or exposure time, or firing angle, etc.), and delivering laser beams accordingly to ablate or dust the second portion of the same target stone. In some examples, multiple different laser sources may be included in the laser system. Stone portions of different compositions may be treated by different laser sources. The appropriate laser to use may be determined by the identification of stone type.

Some features as described herein may be used in relation to a laser system for various applications where it may be advantageous to incorporate different types of laser sources. For instance, the features described herein may be suitable in industrial or medical settings, such as medical diagnostic, therapeutic and surgical procedures. Features as described herein may be used regarding an endoscope, laser surgery, laser lithotripsy, laser settings, and/or spectroscopy.

In the foregoing detailed description, the method and apparatus of the present disclosure have been described with reference to specific embodiments thereof. It will, however, be evident that various modifications and changes may be made thereto without departing from the broader spirit and scope of the present disclosure. The present specification and figures are accordingly to be regarded as illustrative rather than restrictive.

To further illustrate the device and related method disclosed herein, a non-limiting list of examples is provided below. Each of the following non limiting examples can stand on its own or can be combined in any permutation or combination with any one or more of the other examples.

In Example 1, an endoscopic system can comprise: an optical fiber having a distal end extending from a distal end of an endoscope and configured to direct light to and from a target; an interferometer configured to: receive first light pulses from a first frequency comb having a first repetition rate; form reference arm light pulses and measurement arm light pulses from the first light pulses; direct the measurement arm light pulses to and from the target via the optical fiber to form return light pulses; and interfere the return light pulses with the reference arm light pulses to form interferometer output pulses; a beamsplitter configured to interfere the interferometer output pulses with second light pulses from a second frequency comb having a second repetition rate different from the first repetition rate to form system output pulses; an optical detector configured to sense the system output pulses; and processor circuitry configured to: determine, from a time duration between consecutive system output pulses, a spacing between the distal end of the optical fiber and the target; and generate a spacing data signal representing the determined spacing.

In Example 2, the endoscopic system of Example 1 can optionally be configured such that the optical fiber is time-multiplexed to deliver the measurement arm light pulses to and from the target at first times and deliver therapeutic light pulses at second times different from the first times, the therapeutic light pulses being configured to ablate the target, the therapeutic light pulses being spectrally separated from the first light pulses.

In Example 3, the endoscopic system of any one of Examples 1-2 can optionally be configured such that: the optical fiber is further configured to: collect, as collected therapeutic light pulses, at least some of the therapeutic light pulses that are reflected from the target; and direct, as return therapeutic light pulses, at least some of the collected therapeutic light pulses along the optical fiber away from the distal end of the optical fiber; and the endoscopic system further comprises a spectrometer configured to analyze the return therapeutic light pulses.

In Example 4, the endoscopic system of any one of Examples 1-3 can optionally further comprise: the first frequency comb and the second frequency comb, wherein the first frequency comb and the second frequency comb are spaced apart from the endoscope; and wherein the first light pulses and the second light pulses are spectrally separated from the therapeutic light pulses.

In Example 5, the endoscopic system of any one of Examples 1˜4 can optionally further comprise: a therapeutic laser light source spaced apart from the endoscope and configured to generate the therapeutic light pulses at the second times.

In Example 6, the endoscopic system of any one of Examples 1-5 can optionally be configured such that the processor circuitry is further configured to vary at least one operational parameter of the therapeutic laser light source in response to the determined spacing represented by the spacing data signal.

In Example 7, the endoscopic system of any one of Examples 1-6 can optionally be configured such that the processor circuitry is further configured to automatically switch off the therapeutic laser light source when the determined spacing represented by the spacing data signal is less than a specified threshold spacing.

In Example 8, the endoscopic system of any one of Examples 1-7 can optionally further comprise: an actuator configured to advance the optical fiber distally and retract the optical fiber proximally with respect to the endoscope, wherein the processor circuitry is further configured to: compare the determined spacing to a specified threshold; and cause the actuator to automatically reduce a difference between the determined spacing and the specified threshold.

In Example 9, the endoscopic system of any one of Examples 1-8 can optionally be configured such that: the actuator comprises a wheel; the wheel has a center that is fixed in position with respect to the endoscope; the wheel has a circumferential surface that contacts the optical fiber; and the wheel is rotatable from a rotary actuator.

In Example 10, the endoscopic system of any one of Examples 1-9 can optionally further comprise an optical bandpass filter configured to reduce an optical spectrum of the system output pulses.

In Example 11, the endoscopic system of any one of Examples 1-10 can optionally be configured such that: the optical detector is configured to generate an unfiltered electrical signal in response to the sensed system output pulses; the endoscopic system further comprises a low-pass filter configured to reduce high frequency content of the unfiltered electrical signal to form a filtered electrical signal; the endoscopic system further comprises an analog-to-digital converter configured to receive the filtered electrical signal and, in response, generate a digital detector signal; and the processor circuitry is configured to analyze the digital detector signal to determine the time duration between consecutive system output pulses.

In Example 12, the endoscopic system of any one of Examples 1-11 can optionally further comprise: an illumination light source disposed at the distal end of the endoscope and configured to illuminate the target with visible illumination light; a camera disposed at the distal end of the endoscope and configured to generate a video image of the illuminated target; and a display coupled to the processor circuitry and configured to display the video image of the illuminated target and a visual representation of the determined spacing represented by the spacing data signal.

In Example 13, the endoscopic system of any one of Examples 1-12 can optionally be configured such that: the interferometer is a Michelson interferometer; the reference arm light pulses have the first repetition rate; and the measurement arm light pulses are temporally offset from the corresponding reference arm light pulses by a time interval that varies as a function of the spacing between the distal end of the optical fiber and the target.

In Example 14, the endoscopic system of any one of Examples 1-13 can optionally be configured such that the optical fiber is configured such that the measurement arm light pulses enter the optical fiber, propagate to the distal end of the optical fiber, emerge from the distal end of the optical fiber, reflect from the target, enter the distal end of the optical fiber, propagate away from the distal end of the optical fiber, and exit the optical fiber to form the return light pulses.

In Example 15, a method for operating an endoscopic system including an optical fiber having a distal end extending from a distal end of an endoscope can comprise: receiving, with an interferometer, first light pulses from a first frequency comb having a first repetition rate; forming, with the interferometer, reference arm light pulses and measurement arm light pulses from the first light pulses; directing the measurement arm light pulses to and from a target via the optical fiber to form return light pulses; interfering the return light pulses with the reference arm light pulses to form interferometer output pulses; interfering, with a beamsplitter, the interferometer output pulses with second light pulses from a second frequency comb having a second repetition rate different from the first repetition rate to form system output pulses; sensing, with an optical detector, the system output pulses; determining, with processor circuitry, from a time duration between consecutive system output pulses, a spacing between the distal end of the optical fiber and the target; and generating, with the processor circuitry, a spacing data signal representing the determined spacing.

In Example 16, the method of Example 15 can optionally be configured such that: the optical fiber is time-multiplexed to deliver the measurement arm light pulses to and from the target at first times and deliver therapeutic light pulses at second times different from the first times, the therapeutic light pulses being configured to ablate the target, the therapeutic light pulses being spectrally separated from the first light pulses; and the endoscopic system further includes a therapeutic laser light source spaced apart from the endoscope and configured to generate the therapeutic light pulses at the second time.

In Example 17, the method of any one of Examples 15-16 can optionally further comprise: using the processor circuitry to vary at least one operational parameter of the therapeutic laser light source in response to the determined spacing represented by the spacing data signal.

In Example 18, the method of any one of Examples 15-17 can optionally further comprise: using the processor circuitry to automatically switch off the therapeutic laser light source when the determined spacing represented by the spacing data signal is less than a specified threshold spacing.

In Example 19, the method of any one of Examples 15-18 can optionally further comprise: the endoscopic system further includes an actuator configured to advance the optical fiber distally and retract the optical fiber proximally with respect to the endoscope; and the method further comprises using the processor circuitry to: compare the determined spacing to a specified threshold; and cause the actuator to automatically reduce a difference between the determined spacing and the specified threshold.

In Example 20, an endoscopic system can comprise: an endoscope; a therapeutic laser light source spaced apart from the endoscope and configured to generate therapeutic light pulses at first times; a first frequency comb spaced apart from the endoscope and configured to generate first light pulses that repeat at a first repetition rate; a Michelson interferometer configured to split the first light pulses between a reference arm and a measurement arm to form respective reference arm light pulses that repeat at the first repetition rate and measurement arm light pulses that repeat at the first repetition rate; an optical fiber including a distal end extending from the endoscope, the optical fiber configured to: receive the therapeutic light pulses at the first times; receive the measurement arm light pulses at second times different from the first times; direct the therapeutic light pulses and the measurement arm light pulses along the optical fiber to emerge from the distal end of the optical fiber toward a target; collect, as collected light pulses, at least some of the measurement arm light pulses that are reflected from the target; and direct, as return light pulses, at least some of the collected light pulses along the optical fiber away from the distal end of the optical fiber, the Michelson interferometer further configured to interfere the return light pulses with the reference arm light pulses to form interferometer output pulses; a second frequency comb spaced apart from the endoscope and configured to generate second light pulses at a second repetition rate different from the first repetition rate; a beamsplitter configured to interfere the interferometer output pulses with the second light pulses to form system output pulses; an optical detector configured to sense the system output pulses; processor circuitry configured to: determine, from a time duration between consecutive system output pulses, a spacing between the distal end of the optical fiber and the target; and generate a spacing data signal representing the determined spacing; an illumination light source disposed at a distal end of the endoscope and configured to illuminate the target with visible illumination light; a camera disposed at the distal end of the endoscope and configured to generate a video image of the illuminated target; and a display coupled to the processor circuitry and configured to display the video image of the illuminated target and a visual representation of the determined spacing represented by the spacing data signal.

What is claimed is:

1. An endoscopic system, comprising:
an optical fiber having a distal end extending from a distal end of an endoscope and configured to direct light to and from a target;
an interferometer configured to:
receive first light pulses from a first frequency comb having a first repetition rate;
form reference arm light pulses and measurement arm light pulses from the first light pulses;
direct the measurement arm light pulses to and from the target via the optical fiber to form return light pulses; and
interfere the return light pulses with the reference arm light pulses to form interferometer output pulses;
a beamsplitter configured to interfere the interferometer output pulses with second light pulses from a second frequency comb having a second repetition rate different from the first repetition rate to form system output pulses;
an optical detector configured to sense the system output pulses; and
processor circuitry configured to:
determine, from a time duration between consecutive system output pulses, a spacing between the distal end of the optical fiber and the target; and
generate a spacing data signal representing the determined spacing.

2. The endoscopic system of claim 1, wherein the optical fiber is time-multiplexed to deliver the measurement arm light pulses to and from the target at first times and deliver therapeutic light pulses at second times different from the first times, the therapeutic light pulses being configured to ablate the target, the therapeutic light pulses being spectrally separated from the first light pulses.

3. The endoscopic system of claim 2, wherein:
the optical fiber is further configured to:
collect, as collected therapeutic light pulses, at least some of the therapeutic light pulses that are reflected from the target; and
direct, as return therapeutic light pulses, at least some of the collected therapeutic light pulses along the optical fiber away from the distal end of the optical fiber; and
the endoscopic system further comprises a spectrometer configured to analyze the return therapeutic light pulses.

4. The endoscopic system of claim 2, further comprising:
the first frequency comb and the second frequency comb,
wherein the first frequency comb and the second frequency comb are spaced apart from the endoscope; and
wherein the first light pulses and the second light pulses are spectrally separated from the therapeutic light pulses.

5. The endoscopic system of claim 2, further comprising:
a therapeutic laser light source spaced apart from the endoscope and configured to generate the therapeutic light pulses at the second times.

6. The endoscopic system of claim 5, wherein the processor circuitry is further configured to vary at least one operational parameter of the therapeutic laser light source in response to the determined spacing represented by the spacing data signal.

7. The endoscopic system of claim 5, wherein the processor circuitry is further configured to automatically switch off the therapeutic laser light source when the determined spacing represented by the spacing data signal is less than a specified threshold spacing.

8. The endoscopic system of claim 1, further comprising:
an actuator configured to advance the optical fiber distally and retract the optical fiber proximally with respect to the endoscope,
wherein the processor circuitry is further configured to:
compare the determined spacing to a specified threshold; and
cause the actuator to automatically reduce a difference between the determined spacing and the specified threshold.

9. The endoscopic system of claim 8, wherein:
the actuator comprises a wheel;
the wheel has a center that is fixed in position with respect to the endoscope;
the wheel has a circumferential surface that contacts the optical fiber; and
the wheel is rotatable from a rotary actuator.

10. The endoscopic system of claim 1, further comprising an optical bandpass filter configured to reduce an optical spectrum of the system output pulses.

11. The endoscopic system of claim 1, wherein:
the optical detector is configured to generate an unfiltered electrical signal in response to the sensed system output pulses;
the endoscopic system further comprises a low-pass filter configured to reduce high frequency content of the unfiltered electrical signal to form a filtered electrical signal;
the endoscopic system further comprises an analog-to-digital converter configured to receive the filtered electrical signal and, in response, generate a digital detector signal; and
the processor circuitry is configured to analyze the digital detector signal to determine the time duration between consecutive system output pulses.

12. The endoscopic system of claim 1, further comprising:
an illumination light source disposed at the distal end of the endoscope and configured to illuminate the target with visible illumination light;
a camera disposed at the distal end of the endoscope and configured to generate a video image of the illuminated target; and
a display coupled to the processor circuitry and configured to display the video image of the illuminated target and a visual representation of the determined spacing represented by the spacing data signal.

13. The endoscopic system of claim 1, wherein:
the interferometer is a Michelson interferometer;
the reference arm light pulses have the first repetition rate; and
the measurement arm light pulses are temporally offset from the corresponding reference arm light pulses by a time interval that varies as a function of the spacing between the distal end of the optical fiber and the target.

14. The endoscopic system of claim 1, wherein the optical fiber is configured such that the measurement arm light pulses enter the optical fiber, propagate to the distal end of the optical fiber, emerge from the distal end of the optical fiber, reflect from the target, enter the distal end of the optical fiber, propagate away from the distal end of the optical fiber, and exit the optical fiber to form the return light pulses.

15. A method for operating an endoscopic system including an optical fiber having a distal end extending from a distal end of an endoscope, the method comprising:
receiving, with an interferometer, first light pulses from a first frequency comb having a first repetition rate;
forming, with the interferometer, reference arm light pulses and measurement arm light pulses from the first light pulses;
directing the measurement arm light pulses to and from a target via the optical fiber to form return light pulses;
interfering the return light pulses with the reference arm light pulses to form interferometer output pulses;
interfering, with a beamsplitter, the interferometer output pulses with second light pulses from a second frequency comb having a second repetition rate different from the first repetition rate to form system output pulses;
sensing, with an optical detector, the system output pulses;
determining, with processor circuitry, from a time duration between consecutive system output pulses, a spacing between the distal end of the optical fiber and the target; and
generating, with the processor circuitry, a spacing data signal representing the determined spacing.

16. The method of claim 15, wherein:
the optical fiber is time-multiplexed to deliver the measurement arm light pulses to and from the target at first times and deliver therapeutic light pulses at second times different from the first times, the therapeutic light pulses being configured to ablate the target, the therapeutic light pulses being spectrally separated from the first light pulses; and
the endoscopic system further includes a therapeutic laser light source spaced apart from the endoscope and configured to generate the therapeutic light pulses at the second time.

17. The method of claim 16, further comprising:
using the processor circuitry to vary at least one operational parameter of the therapeutic laser light source in response to the determined spacing represented by the spacing data signal.

18. The method of claim 16, further comprising:
using the processor circuitry to automatically switch off the therapeutic laser light source when the determined spacing represented by the spacing data signal is less than a specified threshold spacing.

19. The method of claim 16, wherein:
the endoscopic system further includes an actuator configured to advance the optical fiber distally and retract the optical fiber proximally with respect to the endoscope; and
the method further comprises using the processor circuitry to:
compare the determined spacing to a specified threshold; and
cause the actuator to automatically reduce a difference between the determined spacing and the specified threshold.

20. An endoscopic system, comprising:
an endoscope;
a therapeutic laser light source spaced apart from the endoscope and configured to generate therapeutic light pulses at first times;
a first frequency comb spaced apart from the endoscope and configured to generate first light pulses that repeat at a first repetition rate;
a Michelson interferometer configured to split the first light pulses between a reference arm and a measurement arm to form respective reference arm light pulses that repeat at the first repetition rate and measurement arm light pulses that repeat at the first repetition rate;
an optical fiber including a distal end extending from the endoscope, the optical fiber configured to:
receive the therapeutic light pulses at the first times;
receive the measurement arm light pulses at second times different from the first times;
direct the therapeutic light pulses and the measurement arm light pulses along the optical fiber to emerge from the distal end of the optical fiber toward a target;
collect, as collected light pulses, at least some of the measurement arm light pulses that are reflected from the target; and
direct, as return light pulses, at least some of the collected light pulses along the optical fiber away from the distal end of the optical fiber, the Michelson interferometer further configured to interfere the return light pulses with the reference arm light pulses to form interferometer output pulses;
a second frequency comb spaced apart from the endoscope and configured to generate second light pulses at a second repetition rate different from the first repetition rate;
a beamsplitter configured to interfere the interferometer output pulses with the second light pulses to form system output pulses;
an optical detector configured to sense the system output pulses;
processor circuitry configured to:
determine, from a time duration between consecutive system output pulses, a spacing between the distal end of the optical fiber and the target; and
generate a spacing data signal representing the determined spacing;
an illumination light source disposed at a distal end of the endoscope and configured to illuminate the target with visible illumination light;
a camera disposed at the distal end of the endoscope and configured to generate a video image of the illuminated target; and
a display coupled to the processor circuitry and configured to display the video image of the illuminated target and a visual representation of the determined spacing represented by the spacing data signal.
